(19) United States
(12) Patent Application Publication
(10) Pub. No.: US 2010/0174156 A1
(43) Pub. Date: Jul. 8, 2010

(54) Method of Detecting and Measuring Hydration Imbalance

(76) Inventors: Sohita Patel, Hoffman Estates, IL (US); Dennis Runyan, Salem, WI (US)

Correspondence Address: United States Army Medical Research;and Material Command MCMR-ZA-J, 504 Scott Street, Fort Detrick, MD, 21702-5012, US

(21) Appl. No.: 12/584,920

(22) Filed: Sep. 15, 2009

Related U.S. Application Data
(60) Provisional application No. 61/096,941, filed on Sep. 15, 2008.

(51) Int. Cl. A61B 5/00

(52) U.S. Cl. 600/309

(57) ABSTRACT

Hydration imbalance can adversely affect individuals who work in adverse environments or suffer from hydration related conditions. Rapid and continuous monitoring of at least one saliva component allows monitoring of an individual's hydration level. Interceptive preventive measures based on the detection and measurement of saliva osmolality/osmolarity at the early stages of hydration imbalance negates potential health hazards and death.

This application claims the benefit of priority from provisional application 61/096,941, filed Sep. 15, 2008.

### BACKGROUND OF THE INVENTION

Hydration imbalance-related injuries affect all persons who work in adverse environments or suffer from hydration related conditions. Hydration is also important in military environments, and has a significant impact on mission capabilities of the armed forces. The ability to rapidly detect the hydration levels of military personnel and others would prevent serious health injuries and death, and provide a cost-effective means of maintaining the health of those who are routinely subjected to the impact of hydration on their activities.

Optimal physical performance in normal and adverse environments can be maintained when fluid consumption matches fluid loss. Knowledge of an individual's hydration status assists the individual or health care provider in rectifying body fluid imbalances, provide early detection and rapid assessment of medical conditions and provide accurate indicators of an individual's ability to perform optimally in a given environment.

Hypohydration, also known as dehydration, is a deficiency in water body content. Hypohydration has a negative effect on physical performance as well as on overall health. As noted in Walsh, et al., a strong correlation exists between salivary protein concentration and body mass loss indicating a relation between whole body hydration status and saliva flow rate. See Walsh, Neil P., Saliva Flow Rate, Total Protein Concentration And Osmolality As Potential Markers Of Whole Body Hydration Status During Progressive Acute Dehydration In Humans, 49 Arc. Oral Bio. 149 (2004). A fluid loss of 2% to 3% of total body mass results in heat dissipation, and impacts cardiovascular function and exercise performance. An 8% depletion of total body water causes a stop in saliva flow indicating dehydration. Consequences of dehydration include an elevation in heart rate and core temperature, a decrease in sweating and degradations in performance. Severe dehydration can result in a loss of life.

Hyperhydration, an excess in water body content, can result in severe illness and death. Hyperhydration may lead to hyponatremea, a dangerous condition where the salt concentration of blood is diluted.

The osmolarity of urine has been utilized to measure an individual's hydration status. The correlation between urine color, specific gravity and osmolarity are accurate indices of hydration status. See L. Armstrong, et al., Urinary Indices During Dehydration, Exercise and Rehydration, 8 Int. J. Sport Nutrition 345 (1998); see also L. Armstrong, et al., Urinary Indices Of Hydration Status, 4 Int. J. Sport Nutrition 265 (1994). However, urine testing is inconvenient for the rapid testing of hydration imbalance in field or far forward conditions.

It has also been noted that plasma osmolarity can accurately identify a state of euhydration and is sensitive to changes in hydration during acute dehydration and rehydration. Urine specific gravity and osmolarity are also sensitive to changes in hydration status but lag behind in plasma osmolarity during periods of rapid body fluid turnover and, therefore, correlate only moderately with plasma osmolarity during acute dehydration. See L. Popowski, et al., Blood And Urinary Measures Of Hydration Status During Progressive Acute Dehydration; 33(5) Med Sci Sports Exerc. 747 (2001).

Saliva is a complex fluid. Approximately 99% of saliva is water, and the remaining 1% consists of large organic molecules such as proteins, small organic molecules such as urea, and electrolytes such as sodium and chloride. Whole saliva, defined as the total fluid content of the mouth, contains many other constituents, including serum components, blood cells, bacteria, bacterial products, epithelial cells, cell products, food debris and bronchial secretions. The average daily flow of whole saliva varies in health between 0.75 and 1.5 liters, or more than 20% of total plasma volume. Since virtually all of this volume is reabsorbed in the alimentary canal, saliva secretion plays almost no role in water regulation and electrolyte balance in humans. Saliva is produced by a number of specialized glands that discharge into the oral cavity. Most of the saliva is produced by three pairs of major salivary glands (parotid, submandibular and sublingual), but a small amount is made by the numerous small glands that line the mouth. Percentage contributions of the different salivary glands during unstimulated flow are: 20% from parotid, 65% from submandibular, 7-8% from sublingual, and less than 10% from numerous minor glands. Stimulated high flow rates drastically change percentage contributions from each gland, with the parotid contributing more than 50% of total salivary secretions. Over the years, many attempts have been made to utilize saliva, due to its relative ease of collection, as a biomarker for physiologic, pathologic and biologic testing. These attempts have generally led to less than ideal results for several reasons. Firstly, saliva is produced by three pairs of large glands (parotid, submandibular and sublingual) and many smaller glands found throughout the oral mucosa. The saliva from each of these sources varies greatly in composition, viscosity and quantity. It is impossible to accurately characterize the exact composition of saliva since many factors, including stimulation and flow rate, have a marked effect on salivary composition. However, the parotid gland saliva is consistently and virtually pure serous, and submandibular saliva and sublingual gland saliva is a mixture of mucous and serous saliva. Many studies examining saliva attempt to confound the variability due to the source of the saliva by allowing them to mix in the oral cavity producing “whole” saliva prior to collection. However, these studies are plagued by inadequacy of mixing, varying flow rates from different glands depending on the amount and type of stimulation and contamination. Secondly, the composition of saliva is highly variable and depends on such things as flow rate and source of stimulation. Increasing the flow rate can have a dramatic effect on the composition of the saliva. Artificially stimulated saliva, which is used in most research studies focusing on saliva, may not produce a physiologic composition. Thirdly, saliva is usually contaminated with bacteria, crevicular fluid, food debris epithelial cells and mucin, thereby providing a heterogeneous and variable specimen that depends upon the location within the sample that is harvested. Lastly, analytical methods used for other body fluids, i.e. blood and urine, require novel modifications to provide consistent and reliable results in saliva. Thus, any test of measurements that do not correct for the large variations in salivary flow rates will not be successful as a standardized marker. Since salivary flow rate can be a very difficult measurement to achieve, saliva has had limited usefulness in the past.

It is important to note that there are studies that note the correlation between saliva osmolality and percent body mass loss for the purposes of identifying the state of euhydration (normal state of body water content), and the correlation among saliva osmolality, urine and plasma osmolality. See N. Walsh, et al., Saliva Flow rate, Total Protein Concentration And Osmolality As Potential Markers Of Whole Body Hydration Status During Progressive Acute Dehydration in Humans; 49 Arch. Of Oral Bio. 149 (2004); see also N. Walsh, et al.; Saliva Parameters As Potential Indices Of Hydration Status During Acute Dehydration; Med. & Sci Sports & Exercise 1535 (2004). However, the studies only focus on moderate to severe dehydration in the range of 3% body mass loss (BML). As with every preventive medical procedure, early detection/measurement is extremely important. Walsh, et al., fails to provide early detection means and, due to the complex and heterogeneous nature of saliva discussed above, it is not possible to gauge the correlation of saliva osmolality at low levels of hydration imbalance (either hyperhydration or hypohydration).

The present invention overcomes the prior art as discussed below.

### BRIEF DESCRIPTION OF THE FIGURES

FIG. 1 shows changes in plasma Osm between pre-exercise, post-exercise and following rehydration;

FIG. 2 shows changes in saliva Osm between pre-exercise, post-exercise and following rehydration;

FIG. 3 shows percentage changes body mass loss during dehydration v. saliva Osm for males; and

FIG. 4 shows the combined data of Sosm baseline data compared with Posm and Uosm.

### SUMMARY OF THE INVENTION

It is an objective of the present invention to provide a non-invasive method of determining hydration levels of an individual.

It is also an objective of the present invention to provide a rapid hydration marker that is capable of continuously monitoring at least one saliva component.

It is also an objective of the present invention where the saliva component is salivary osmolality/osmolarity. These and other objectives are described below.

### DETAILED DESCRIPTION OF THE EMBODIMENTS

The present invention is directed to a non-invasive method of determining hydration levels of an individual. It is important to note that the present invention is not limited to the detection of hypohydration or hyperhydration, but to the rapid and continuous monitoring of body hydration levels to detect for hydration imbalance. The method of the present invention may utilize a miniature microfluidic salivary monitor to determine the hydration levels in the user without interfering with speech, respiration and deglutition.

The present invention utilizes saliva as a rapid hydration marker and continuously monitors at least one saliva component in a user to determine hydration levels. This method is particularly effective in determining hydration levels in athletes, first responders, armed forces, combat casualty situations and for others users where the risk of hydration imbalance is prevalent. Early detection and intervention by health care providers provides necessary hydration therapy without resulting in serious injuries or the unnecessary loss of life.

The method of the present invention provides rapid and continuous monitoring of at least one salivary component. The method also allows for the subsequent transmission of that data to a remote receiver, which allows remote monitoring of a user's hydration level by health care providers.

As stated above, the method of the present invention measures at least one salivary component to determine hydration. In accordance with the present invention, the term “salivary component” refers to the physical characteristics of saliva and not components of saliva. These components include, but are not limited to, salivary osmolality, salivary osmolarity, salivary amylase and total salivary protein. In a preferred embodiment, the present invention is directed to the measurement of salivary osmolality.

Salivary osmolality presents colligative properties that are not quantity dependent or dependent on what type molecules are in solution. Thus, salivary osmolality reflects overall hydration levels without relevance to the amounts of saliva being produced or the composition of the saliva. In accordance with the present invention, it is critically important to correlate the osmolality of pure parotid saliva with the osmolality of whole saliva to detect hydration imbalances and provide rapid diagnosis of early and mild dehydration, thus making it simple to intervene and reverse its negative sequelae.

In accordance with the present invention, saliva provides the advantages over blood or urine because saliva can be collected noninvasively, multiple specimens may be collected from the same individual at optimum times for diagnostic use or specimens may be collected continuously, and the collection of samples does not require trained professionals and can be conducted at remote sites and saliva remains stable at ambient temperatures for several weeks.

For the purposes of the present invention, salivary osmolality (parotid or whole saliva) is a measure of the number of dissolved particles per unit of water in saliva. In a solution, the fewer the particles of solute in proportion to the number of units of water (solvent), the less concentrated the solution. Because osmotic equilibrium is constantly being maintained on either side of the cell membrane (homeostasis), measurement of the saliva osmolality provides hydration status within the cells. In response to the osmotic pressure being exerted by the molecules of solute in the intracellular and extracellular fluids, water moves freely back and forth across the membranes in the salivary glands.

Salivary osmolality reflects the status of hydration of the intracellular as well as the extracellular compartments and thus describes total body hydration. A low saliva osmolality is indicative of a higher than usual amount of water in relation to the amount of particles dissolved in it. Thus, low saliva osmolality accompanies overhydration, or edema, and an increased saliva osmolality accompanies a state of fluid volume deficit or dehydration.

As will be understood by one of ordinary skill in the art, the detection of hydration imbalance utilizing salivary osmolality includes salivary osmolarity. In dilute solutions where the solvent is water having a density of 1 g/ml, such as saliva, osmolality and osmolarity are equal.

In accordance with the present invention, whole saliva osmolality (Sosm) and parotid saliva osmolality (Posm) measurements were utilized for detecting and monitoring hydration imbalance. In order to be utilized as a marker for total body hydration, Sosm must (1) provide a “normal” range in humans that allows for the development of a quantitative hydration index such that salivary osmolalities lying above this normal range are indicative of dehydration, or (2) there is a consistent individual base level range of salivary osmolality in euhydrated individuals such that each human subject establishes a minimum change indicative of dehydration. Both situations require that dehydration salivary osmolalities return to normal or base levels once rehydration is accomplished. Based upon test results, the consistency in the results of Sosm makes it a screening measurement that allows prevention of moderate to severe dehydration.

Test 1:

Based on recommendations from USARIEM subject matter experts, weight measurements within one standard deviation from an established mean weight (6 separate baselines weights on 6 different days) were employed in the previous study to establish euhydration. All subjects met the weight standard for euhydration. Our study revealed this not to be a reliable method to establish euhydration. By using the widely accepted euhydration range for Posm of 288+/−4 mOsm/kg, 20 of 39 subjects were, by this definition, dehydrated with Posm above 292 mOsm/kg, and 1 subject was hyperhydrated with a Posm below 284 mOsm/kg. It is possible that the large percentage of subjects who were already mildly dehydrated prior to exercise added an increased variability to the results. A second method to establish euhydration was to prescribe a known amount of water (30 ml/kg of body weight) the day prior to exercise. One of the goals of this study was to determine whether the second method provided a more predictable method of establishing euhydration. On the day prior to exercising, each subject was asked to drink 30 ml of water for every kg of their body weight. Except for drinking this prescribed water, the rest of this exercise phase was the same as the first stage. Active Duty male military personnel volunteers were used. The volunteers were asked to exercise under hot conditions (40 degrees C. with 20% humidity), to produce a mild hypohydrated state, in approximately the 1-2% range. The volunteers exercised for 60 minutes with samples being collected prior to exercise, following exercise and following rehydration. All subjects sat for 30 minutes in a temperate environment prior to collection of blood and saliva samples. Subjects were allowed to stand to provide a urine sample and weighing. Post exercise samples were collected after the volunteer sat undisturbed and not consuming any food or drink for 30 minutes following their 60 minute exercise session. The post hydration samples were collected following an additional 30 minutes of sitting while drinking water ad libitum. The osmolality of the saliva samples was correlated with the osmolality of the plasma and urine samples, and the specific gravity of the urine samples. The stage 2 exercise day occurred 3-7 days following the stage 1 exercise day to allow full volunteer recovery. The results showed that there are significant differences between pre-exercise Sosm and post-exercise Sosm (p<0.001) following a 1.1-3.06 BML.

In order to determine if osmolality changes in Posm during dehydration differs from that of Sosm, all participating individuals must be in an euhydrated state prior to the dehydration thermal exercise process. Male volunteers between the ages of 18 and 45 underwent a study analysis following established protocol. While women and the effects of the menstrual cycle on salivary osmalality were considered, the testing is incomplete at this time. However, it is within the scope of the present invention that the method applies to both genders.

Test 2: Collection of Urine, Blood, Parotid Saliva and Whole Saliva

During baseline visits, blood, urine, parotid saliva and whole saliva samples were collected. A first exercise period occurred no later than 14 days subsequent to a first baseline collection visit (that established euhydration and other baseline qualifications). Pre-exercise whole saliva (at least 1.0 ml), parotid saliva (at least 0.5 ml), blood samples (3.0 ml) and urine samples (at least 1.0 ml) were collected, and the volunteers exercised for 60 minutes at 3.5 miles per hour at a 6% grade to expend approximately 350 Watts in an environmental chamber (40 degrees C. and 20% humidity). Subsequent to the exercise period and a 30 minute rest period, blood samples (at least 3 ml), whole saliva (at least 1.0 ml), parotid saliva (at least 0.5 ml) and an optional urine sample (at least 1.0 ml) were collected. A second exercise visit 3-7 days past the first exercise visit was conducted. The exercise protocol remained the same as for the first exercise visit. On each exercise day, samples of whole saliva, parotid saliva, blood and urine were collected three times. Once before exercise, after 60 minutes of exercise and after 30 minutes of recovery time. To collect whole saliva, the saliva was allowed to accumulate in the mouth for 2 minutes and the saliva expectorated into a test tube. Porotid saliva was collected from both parotid glands simultaneously in intraoral Schaeffer cups and combined prior to osmolality testing.

Data Analysis of Test 2:

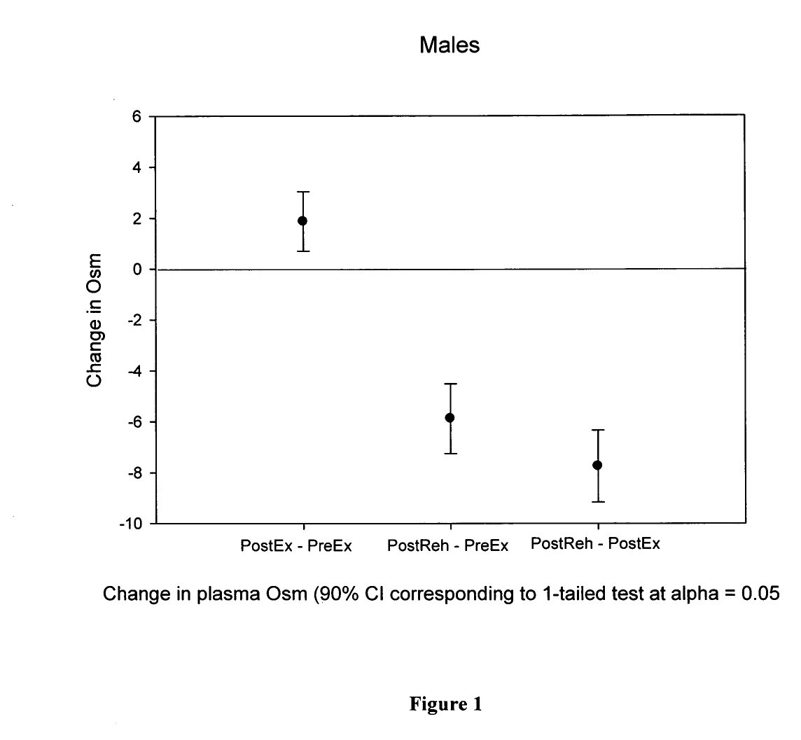

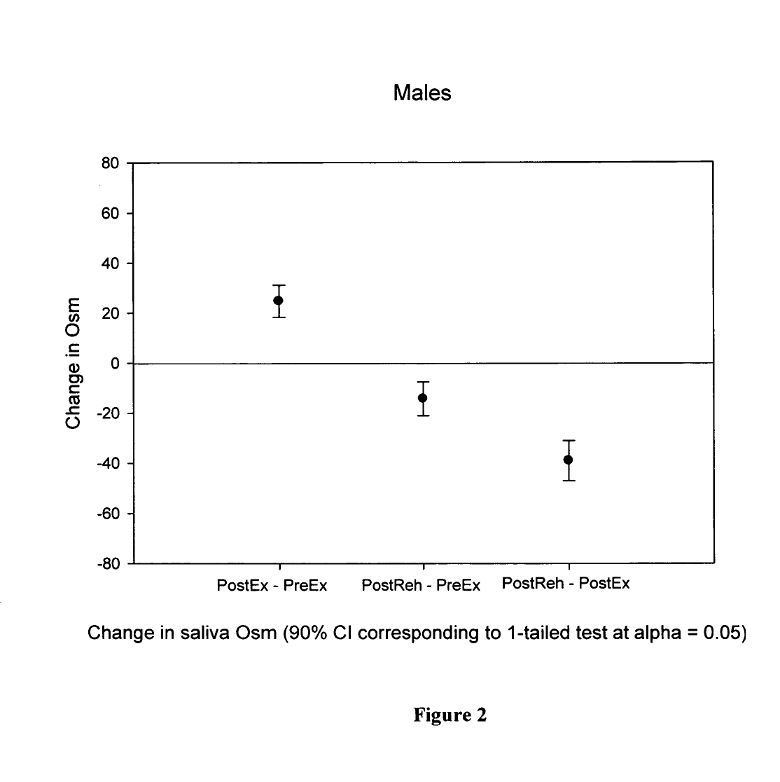

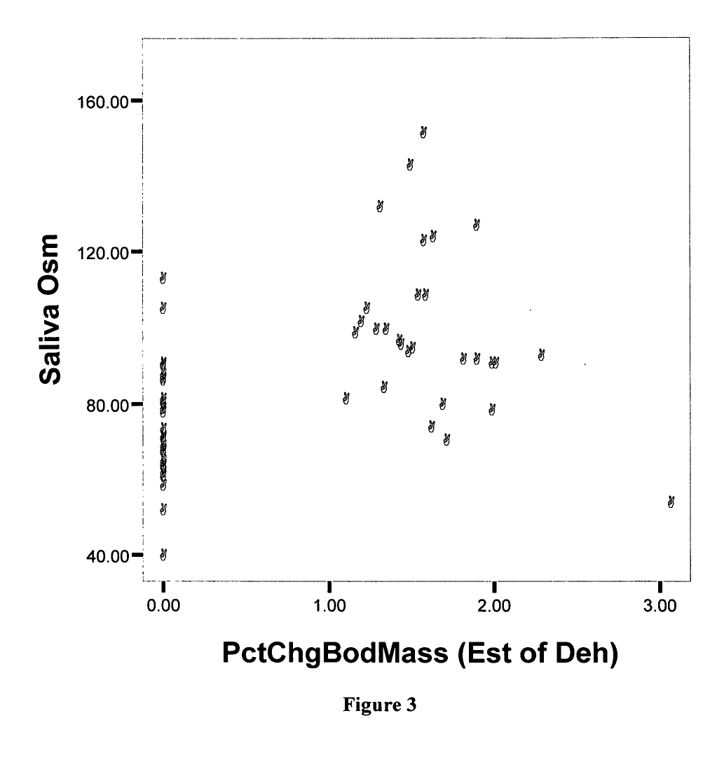

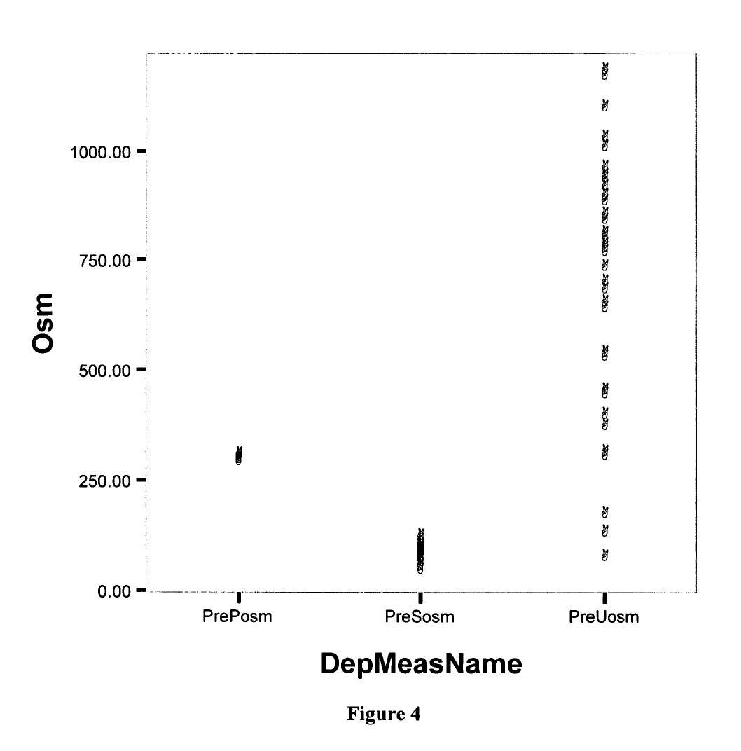

In addition to blood and urine, measures of Posm, Sosm, and parotid saliva were made in triplicate and averaged. In addition to standard statistical corrections, bivariate linear regressions were calculated for changes in Posm, Sosm and parotid saliva. As shown in FIG. 1, Posm significantly increased post-exercise relative to pre-exercise (p=0.002, 1-tailed test), decreased post-rehydration relative to pre-exercise (p=0.001, 1-tailed test) and decreased post-rehydration relative to post-exercise (p=0.001, 1-tailed test). Similarly, as shown in FIG. 2, Sosm significantly increased post-exercise relative to pre-exercise (p=0.001, 1-tailed test), decreased post-rehydration relative to pre-exercise (p=0.001, 1-tailed test) and decreased post-rehydration relative to post-exercise (p=0.001, 1-tailed test). As shown in FIG. 3, correlations between changes in Sosm and body mass were significant, with change in Sosm accounting for 17% of the variation in change in Posm, and approached statistical significance with percent change in body mass. As shown in FIG. 4, all individual data for Posm, Sosm, and Uosm from pre-exercise (baseline) conditions shows the central tendencies and relative variation among these dependent measures for the entire data set. As can be seen, data from Posm and Sosm are much more tightly grouped than for Uosm.

The present invention is directed to the early detection/measurement of hydration imbalance. Early detection/measurement allows for early intervention to prevent significant advancement of a disease or condition. For this reason, early detection of the mild to moderate range of hydration imbalance, particularly dehydration, is the most critical range when addressing prevention of injuries. It allows for self rehydration prior to the onset of conditions where medical treatment is required.

Using the methods of the present invention, whole and/or parotid saliva can be collected. The osmolality of the saliva can then be measured utilizing a freezing point depression osmometer and the individual's hydration level can be determined. In accordance with the present invention, a Sosm value that is 40% above their baseline Sosm indicates dehydration. By continuing to monitor Sosm and rehydrating the individual, euhydration range of Sosm can be achieved within short periods of time, preferably within 30 minutes.

What is claimed is:

1. A method for non-invasively detecting hydration imbalance comprising utilizing saliva as a hydration marker, and continuously monitoring at least one salivary component.

2. A method as recited in claim 1, wherein said at least one salivary component is selected from the group consisting of salivary osmolality, salivary osmolarity, salivary amylase and total salivary protein.

3. A method as recited in claim 1 wherein said at least one salivary component is salivary osmolality.

4. A method as recited in claim 3, wherein said osmolality measurements further comprises whole saliva osmolality and parotid saliva osmolality.

5. A method as recited in claim 4, wherein said whole saliva osmolality and parotid saliva osmolality measurements further comprise measuring baseline euhydration osmolality levels.

6. A method as recited in claim 5, wherein said whole saliva osmolality and parotid saliva osmolality measurements further comprise measuring pre-exercise and post-exercise osmolality levels.

7. A method as recited in claim 6, wherein said euhydration osmolality levels, said pre-exercise and post-exercise osmolality levels further comprise changes in said levels so as to indicate hydration imbalance.

8. A method as recited in claim 7 wherein said hydration imbalance further comprises measuring hyperhydration.

9. A method as recited in claim 7, wherein said hydration imbalance further comprises measuring hypohydration.

10. A method as recited in claim 9 wherein a value of 40% above said baseline of said whole saliva measurement indicates dehydration.
